(19) United States
(12) Patent Application Publication
(10) Pub. No.: US 2011/0237677 A1
(43) Pub. Date: Sep. 29, 2011

(54) Inhibitors

(75) Inventors: Joelle Alcock, Nottingham (GB); Joanne Lymn, Nottingham (GB); Raheela Khan, Nottingham (GB)

(73) Assignee: The University of Nottingham, Nottingham (GB)

(21) Appl. No.: 12/997,424

(22) PCT Filed: Jun. 10, 2009
(86) PCT No.: PCT/GB2009/001455

§ 371 Date: Jun. 9, 2011

(30) Foreign Application Priority Data
Jun. 12, 2008 (GB) 0810706.2
Feb. 18, 2009 (GB) 0902661.8

(51) Int. Cl. A61K 31/18; A61K 31/145; A61P 15/00; A61P 15/06

(52) U.S. Cl. 514/603; 514/665

(57) ABSTRACT

The invention relates to inhibitors of tissue transglutaminase II activity and their use as a tocolytic agent.

### CROSS-REFERENCE TO RELATED APPLICATIONS

This application is a U.S. National Phase of PCT Patent Application No. PCT/GB2009/001455 filed Jun. 10, 2009 and claims priority to United Kingdom Patent Application Nos. GB 0810706.2 filed Jun. 12, 2008 and 0902661.8 filed Feb. 18, 2009, the disclosures of which are incorporated herein by reference in their entirety for all purposes.

### FIELD OF THE INVENTION

The invention relates to inhibitors of tissue transglutaminase II and their use as a tocolytic agent.

### BACKGROUND TO THE INVENTION

Preterm delivery, judged to be delivery prior to 37 weeks gestation, remains a major problem in obstetrics affecting 6-15% of all deliveries with 75% of all perinatal deaths occurring in premature infants and for a variety of complex reasons the frequency of preterm birth seems to actually be increasing. While advances in technology have resulted in a decrease in perinatal mortality there has been a corresponding increase in both short and long-term morbidities. Initially preterm birth may require long periods in intensive care for the baby. The NHS reportedly spends nearly £40K on each baby weighing <1000 g, with over £70 million being spent each year for neonatal intensive care. In the longer term preterm birth has been associated with a number of chronic health problems including deafness, blindness and cerebral palsy. These conditions undoubtedly impact on the lifestyle of both the infants themselves and their parents. The indirect economic costs are incalculable arising as a result of, for example, loss of employment to care for a preterm baby.

The intractability of the problem of preterm labour arises because despite substantial research efforts over the past decade the processes leading to parturition in women remain obscure. While we can identify a number of labour associated proteins, including up-regulation of the oxytocin receptor, we are less clear in terms of the associated signalling mechanisms. Indeed until we understand more about the signalling mechanisms responsible for the onset of normal, term, labour we cannot judge whether spontaneous preterm labour is the result of the premature activation of these pathways or whether it results from different signalling mechanisms.

There are two main groups of drugs licensed as myometrial relaxants or tocolytics.

Oxytocin receptor antagonists (Atosiban)

Beta2-adrenoceptor agonists (ritodrine, terbutaline, salbutamol)

The dihydropyridine calcium channel blocker (nifedipine) and the cyclooxygenase inhibitor (indometacin) can also used as myometrial relaxants (unlicensed indication). Progesterone may also be used.

Tocolytics currently available are only able to delay labour for around 48-72 hours and this time-period allows for transfer of the mother to a more appropriate facility and the administration of steroids to develop the baby's lungs. The current tocolytics also have a number of adverse effects for both the mother, including effects on the cardiovascular system as well as hyperglycaemia, hypokalaemia and bronchospasm, and the baby, including impaired renal function, fetal tachycardia and hypo- or hyperglycemia at birth.

Many of the current tocolytics act on cell-surface receptors either as antagonists at the oxytocin receptor, to reduce the contractile response, or as agonists at beta2-adrenoceptors, to stimulate a relaxation of the myometrium. These receptors can be up-regulated/desensitized over time and hence the tocolytic effects overcome. Moreover, current tocolytics may have deleterious side effects for mother and baby. The present invention seeks to address the problems associated with current tocolytics.

Eight distinct transglutaminases have been identified in mammals including the ubiquitously expressed tissue transglutaminase II (tTGII). tTGII has been demonstrated to exist in two mutually exclusive modes of function, firstly as a transglutaminase, catalysing protein cross-linking, and secondly as a GTPase where it is more commonly known as the high molecular weight G protein Gh. Thus tTGII acts as a bifunctional enzyme, having both GTPase and transglutaminase activity and plays a key role as a signal transducer through either the hydrolysis of GTP or by the reorganising of a protein-protein relationship. Exchange of GDP for GTP by tTGII/Gh is facilitated by the activation of a number of cell surface receptors, including α-adrenergic and oxytocin receptors, by contractile agonists. Moreover up-regulation of Gh expression has been demonstrated to occur throughout a rat model of pregnancy reaching peak levels at term.

Inhibitors of tTGII have shown promise in the treatment of neurodegenerative diseases, celiac sprue and certain types of cancer (Siegel and Khosla, Pharmacol Ther. 115(2): 232-245, 2007). However, their use as a tocolytic has not been previously described.

### STATEMENTS OF THE INVENTION

The present invention is based on the finding that inhibitors of transglutaminase activity, in particular transglutaminase II, are useful in the treatment of preterm labour. Thus according to a first aspect of the invention there is provided the use of a transglutaminase antagonist, in particular transglutaminase II (tTgII), as a tocolytic agent. Tissue transglutaminase II is an intracellular molecule involved in signal transduction downstream of the receptors and channels normally targeted and inhibition of this enzyme is more likely to cause prolonged cessation of the contractile signalling pathway. Administration of a tissue transglutaminase inhibitor may result in stopping contractions in a more controlled manner for longer periods compared with the shorter timescale achieved by current tocolytic agents. Moreover, in normal physiological conditions tissue transglutaminases tend to be inactive and consequently inhibition is unlikely to cause side-effects as repercussion of altering other systems within the body.

The transglutaminase antagonist may include small inhibitory or interfering RNA (siRNA), antibodies (for example antibody fragments/Fab fragments), small organic molecules, (for example peptides, cyclic peptides), and dominant negative variants of tTgII.

Preferably the antagonist is an inhibitor of transglutaminase II (tTgII) activity.

Preferably the inhibitor is an amine. The amine may be a monoamine or a polyamine for example a diamine or triamine. It is preferred that the amine is not a tetramine.

In one embodiment of the invention the amine is a monoamine including, for example, cysteamine.

In one embodiment of the invention the amine is a diamine including, for example, putrescine.

The amine may be a polyamine comprising two or more amino groups, for example 2 or 3 amino groups, wherein the amino groups are primary or secondary amino groups. In one embodiment of the invention the inhibitor comprises two or more, for example 2 or 3 primary amino groups. In an alternative embodiment of the invention, the inhibitor comprises two or more, for example 2 or 3, amino groups which are a mixture of primary and secondary amino groups.

Preferably the amine is a sulphur containing amine. For example the amine may be selected from cysteamine and cystamine. The amine may comprise one or more sulphur atoms for example 1, 2 or 3 sulphur atoms, two or more of which may be linked to form a disulphide linkage. The amine may further comprise oxygen for example 1 or 2 oxygen atoms.

Preferably the amine comprises 2 sulphur atoms. This in one embodiment of the invention, the amine is cystamine.

The amine may be an aminothiol. Thus, in one embodiment of the invention, the amine is cysteamine.

Preferably the amine comprises less than 4 amino groups, for example, the amine may comprise 1, 2 or 3 amino groups. The amine may be selected from the group consisting of cadaverine, putrescine, cystamine, cysteamine, spermidine and histamine. The cadaverine may be a substituted cadaverine for example selected from monodansyl cadaverine and biotin cadaverine. Preferably the cadaverine is monodansyl cadaverine.

Preferably still the amine consists of less than 4 amino groups, for example, the amine consists of 1, 2 or 3 amino groups.

It is preferred that the amine is not spermine.

The amine may comprise at least 2 carbon atoms for example at least 4 carbon atoms. The amine may comprise at least 5 carbon atoms, for example between 5 and 9 carbon atoms. In one embodiment of the invention the amine comprises 5 carbon atoms.

In a preferred aspect of the invention the inhibitor is a competitive inhibitor of tTgII. Preferably still the inhibitor is a competitive amine inhibitor of tTgII. Competitive amine inhibitors inhibit tTgII activity by competing with natural amine substrates, such as protein bound lysine residues, in the transamidation reaction that it catalyses. Thus tTgII is still enzymatically active and transamidation continues to occur in the presence of competitive amine inhibitors.

Preferably the tocolytic agent is useful in the treatment or prevention of disorders originating in uterine contractions. Thus the invention further provides the use of an inhibitor of tTgII in the manufacture of a medicament for the treatment or prevention of disorders originating in uterine contractions. The disorders may include any disorder where a cessation, either complete or partial, in uterine contractions is required to treat or prevent the disorder. The disorder may include preterm labour in pregnant females and dysmenorrhea in non-pregnant females.

Although we suggest that an inhibitor of tTgII may cause a complete cessation of contractions rather than purely a delay we also suggest that this effect could be reversible and unless the required dose is maintained contractions will ultimately resume. This is important therapeutically, allowing normal labour to progress when medical conditions are more favourable or, in the case of a contraindication, on return to normal physiological conditions. Thus in a preferred aspect of the invention the effect of the tTgII inhibitor on uterine contractions is reversible.

A further aspect of the invention provides the use of an amine, or pharmaceutically acceptable salt thereof, in the manufacture of a medicament for the treatment or prevention of disorders originating in uterine contractions.

The invention further provides a method for the treatment, prevention or delay of progression of preterm labour, which comprises administering to a patient a therapeutically effective amount of an inhibitor of tTgII, for example an amine. Preferably the amount administered is sufficient to maintain a cessation of uterine contractions in the subject until such time as it is desirable to allow the contractions to resume, for example where labour is intended, in which case the amount of inhibitor administered is either reduced or stopped.

The invention further provides a pharmaceutical formulation comprising an inhibitor of tTgII for use as a tocolytic agent. The formulation may include an inhibitor of tTgII, alone or in combination with one or more other tocolytic agents in an amount effective to inhibit or counter the onset of uterine contractions. Such tocolytic agents include progesterone, beta-adrenoreceptor stimulants such as epinephrine or its synthetic analogs and derivatives salbutamol, terbutaline, isoxsuprine, ritodrine, and fenoterol, magnesium sulfate, ethanol, activin antagonists, cardiac antiarrhythmics such as lidocaine or ocamide, nitric oxide donors such as S-nitroso-N-acetylpenicillamine, nitric oxide nucleophiles and adducts, nitroglycerin, hydroxylamine, sodium azide, diethylamino nitric oxide and analogs, and nitric oxide precursors such as L-arginine, and calcium channel-blocking agents such as nipedifine or nicardipine. A method of the invention may provide for the administration of an inhibitor of tTgII and another pharmaceutical agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

Where appropriate, inhibitors of the invention may be in the form of pharmaceutically acceptable salts. The term “pharmaceutically acceptable” as used herein means a non-toxic material that does not interfere with the effectiveness of the biological activity of the active ingredients.

Pharmaceutically acceptable salts can be synthesized from the parent compound which contains a basic or acidic moiety by conventional chemical methods. Generally, such salts can be prepared by mixing the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the inhibitors wherein the parent compound is modified by making acid or base salts thereof. For example, the conventional non-toxic salts or the quaternary ammonium salts which are formed, e.g. from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention also encompasses therapeutically effective derivatives of the inhibitors which retain the biological activity of the inhibitor and are useful as a tocolytic.

Besides being useful for human treatment, the tTgII inhibitors may be useful for veterinary treatment of mammals, including companion animals and farm animals, such as, but not limited to, horses, dogs, cats, cows, sheep and pigs.

As used herein the term “inhibitor” refers to any species which retards, blocks or prevents an interaction, for example a tTgII-ligand interaction. Typically, inhibition does not result in 100% blockage but rather reduces the amount and/or speed of interaction.

As used herein, the term “treatment” includes partial or total inhibition of uterine contractions.

As used herein, the term “prevention” includes either preventing the onset of clinically evident preterm labour altogether or preventing the onset of a preclinically evident stage of preterm labour in individuals at risk.

The phrase “therapeutically-effective” is intended to qualify the amount of inhibitor, for example amine, which will achieve the goal of improvement in severity and the frequency of incidence over treatment of each agent by itself, while avoiding adverse side effects typically associated with alternative therapies.

The term “subject” for purposes of treatment includes any human or animal subject and preferably is a human subject. For methods of prevention, the subject is any human or animal subject, and preferably is a human subject who is currently pregnant and at risk for experiencing preterm labour.

As used herein the expression “preterm labour” includes the onset of labour prior to the full gestation period which is usually 37 weeks.

The active compounds of the present invention may be administered by any suitable route known to those skilled in the art, preferably in the form of a pharmaceutical composition adapted to such a route, and in a dose effective for the treatment intended. The active compounds and composition may, for example, be administered orally, intravascularly, intraperitoneally, intranasal, intrabronchial, subcutaneously, intramuscularly or topically (including aerosol).

For oral administration, the pharmaceutical composition may be in the form of; for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose; mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

For rectal administration, the active ingredient may be formulated into suppositories using bases which are solid at room temperature and melt or dissolve at body temperature. Commonly used bases include cocoa butter, glycerinated gelatin, hydrogenated vegetable oil, polyethylene glycols of various molecular weights, and fatty esters of polyethylene stearate.

The dosage form and amount can be readily established by reference to known preterm labour treatment or prophylactic regiments. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

According to a further aspect of the invention there is provided a method to screen for a tocolytic agent that modulates, for example inhibits, the activity of a polypeptide having the sequence shown in FIG. 1, or a variant polypeptide thereof, wherein the method comprises the steps of:

(i) forming a preparation comprising a polypeptide, or sequence variant thereof, and at least one agent to be tested; and
(ii) determining the activity of said agent with respect to the activity of said polypeptide.

As used herein the term “variant” is intended to encompass polypeptides which although not identical in sequence to the polypeptide of FIG. 1, have transglutaminase activity, specifically tTgII activity. In a method of the invention said agent may be an antagonist. Agents identified by the screening method of the invention may include siRNA, antibodies, small organic molecules, (for example peptides, cyclic peptides), and dominant negative variants of the polypeptides herein disclosed.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The invention will now be described by way of example only with reference to the following Figures in which:

FIG. 1 Protein sequence of TGM2 (tissue-type transglutaminase II/G-alpha H/Gh)

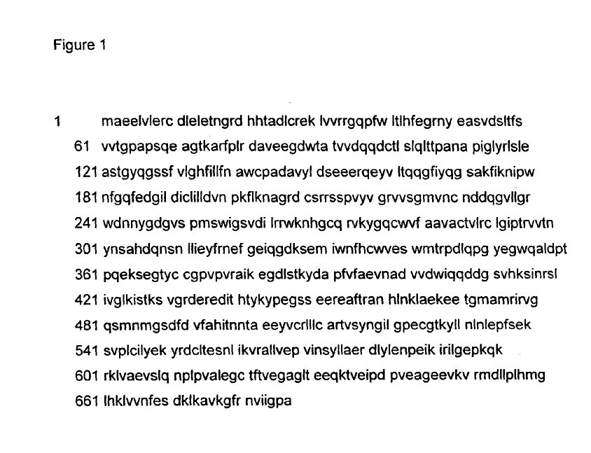

FIG. 2 Oxytocin-stimulated contractions in myometrial strips are attenuated by cystamine. Following flushing with physiological salt solution contractions resumed to control levels. An additional dose of 10⁻²M cystamine following flushing reduced contractions to 14%±3.7 (n=7) of control myometrium. Tissue is used 24 hours following elective caesarean and contractions initially stimulated with 10⁻¹⁰M oxytocin. A) Each experiment shown separately. B) Meaned experiments demonstrating a LogEC50 of −2.884. Contractions are significantly different from controls at cystamine concentrations of 10⁻³M and 10⁻²M with P<0.05 and P<0.0005, respectively.

FIG. 3 Diagrammatic representations showing that oxytocin-induced contractions in myometrial strips are inhibited by MDC in a dose-dependent manner. Contractions are significantly different from controls at 10⁻⁵M and 10⁻⁴M MDC with P<0.05 and P<0.0001 respectively, (n=5). Tissue is used 24 hours following elective caesarean section and contractions are initially stimulated with 10⁻¹⁰M oxytocin. A) Each experiment shown separately (dotted lines) with control (unbroken line). B) Experiments pooled demonstrating a LogEC₅₀ of −4.657. C) Representation of a typical isometric trace where first arrow indicates addition of 10⁻⁴M MDC and second arrow indicates washing with physiological salt solution and re-stimulation with oxytocin 10⁻¹⁰M.

FIG. 4 Diagrammatic representations showing that bradykinin- and phenylephrine-induced contractions in myometrial strips are inhibited by both cystamine and MDC in a dose-dependent manner. Tissue is used 24 hours following elective caesarean section. A) Bradykinin induced contractions (10⁻⁸M) are inhibited in a dose dependent manner by both cystamine (n=6, significant at 10⁻⁵M P<0.05) and MDC (n=1) when compared to control myometrial strips (n=5). B) Phenylephrine induced contractions (10⁻⁸M) are inhibited in a dose dependent manner by both cystamine (n=3) and MDC (n=1) when compared to control myometrial strips (n=2).

FIG. 5 Diagrammatic representations showing that oxytocin mediated a concentration dependent increase in intracellular calcium concentration, which is affected following pre-incubation with either cystamine (A, B) or MDC (C, D). Cells are harvested from myometrial biopsies following enzyme dissociation at passage 0 or 1 plated in 96-well plates and incubated in media containing a fluorescent calcium indicator. Fluorescent fluctuations were measured on FlexStation (Molecular Probes) following oxytocin stimulation with or without pre-incubation for 10 minute with either cystamine 10⁻⁶M, n=6 or MDC 10⁻⁶M, n=6. Maximal calcium mobilisation was significantly reduced following incubation with (B) cystamine (Emax control 121.1, +cystamine 87.67 P<0.05) and (D) MDC (Emax control 98.61, +MDC 81.45 P<0.01).

FIG. 6 Diagrammatic representations showing that oxytocin-induced contractions in myometrial strips are inhibited by cysteamine in a dose-dependent manner. Contractions are significantly different from controls at 10⁻³M with P<0.01 (n=5). Tissue is used 24 hours following elective caesarean section and contractions are initially stimulated with 10⁻¹⁰M oxytocin. A) Each experiment shown separately (dotted lines) with control (unbroken line). B) Experiments pooled demonstrating a LogEC₅₀ of −5.789. C) Representation of a typical isometric trace where each arrow indicates cumulative additions of cysteamine (10⁻⁹M-10⁻³M) and final arrow indicates washing with physiological salt solution and re-stimulation with oxytocin 10⁻¹⁰M.

FIG. 7 Diagrammatic representations showing that oxytocin mediated a concentration dependent increase in intracellular calcium concentration, which is affected following pre-incubation with cysteamine (A, B). Cells are harvested from myometrial biopsies following enzyme dissociation at passage 0 or 1 plated in 96-well plates and incubated in media containing a fluorescent calcium indicator. Fluorescent fluctuations were measured on FlexStation (Molecular Probes) following oxytocin stimulation with or without pre-incubation for 10 minute with cysteamine 10⁻⁶M, n=5. B) Maximal calcium mobilisation was significantly reduced following incubation with cysteamine (Emax control 114, +cystamine 98.95*P<0.05).

FIG. 8 Diagrammatic representations showing that oxytocin-induced contractions in myometrial strips are inhibited by putrescine in a dose-dependent manner. Tissue is used 24 hours following elective caesarean section and contractions are initially stimulated with 10⁻¹⁰M oxytocin. A) Each experiment shown separately (dotted lines) with control (unbroken line). B) Experiments pooled demonstrating a LogEC50 of −4.247, n=4.

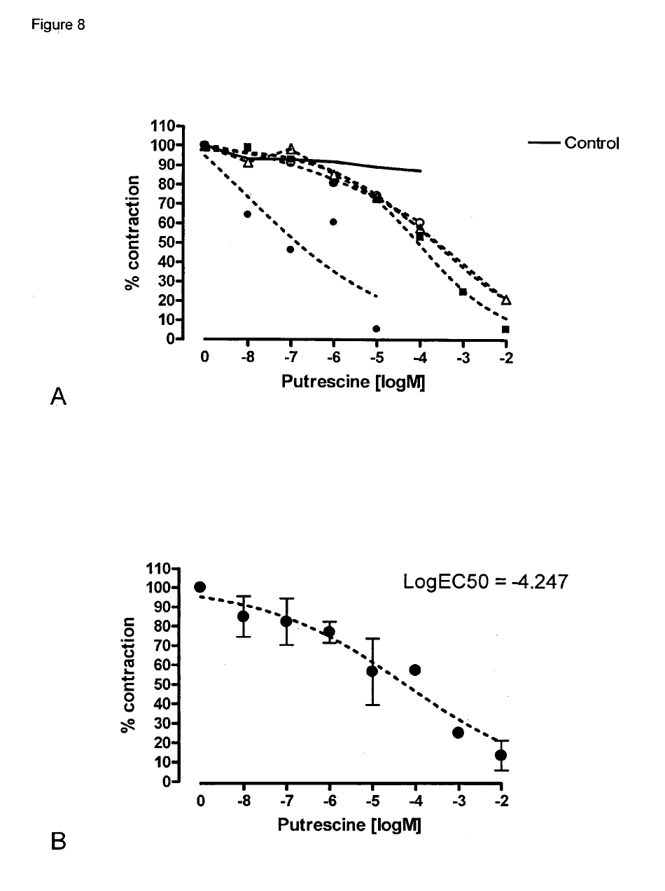

FIG. 9 Diagrammatic representations showing that spontaneous contractions in myometrial strips are inhibited by cystamine (n=4), cysteamine (n=3) and MDC (n=2) in a dose-dependent manner. Control strips are shown with unbroken line (n=2). Tissue is used 24 hours following elective caesarean section and contractions are allowed to spontaneously initiate under 2 g tension and in a temperature controlled and oxygenated organ bath.

### EXAMPLES

### Materials and Methods

### Isometric Tension Recording:

Longitudinal myometrial strips were mounted in an organ bath and contractile activity recorded as a measure of tensile force as previously described (Chanrachakul, Broughton-Pipkin et al., Am J Obstet Gynecol 192(2): 458-63, 2005). Myometrial contractions were initially stimulated by addition of oxytocin, bradykinin or phenylephrine (Sigma-Aldrich; 10⁻¹⁰ mol/L, 10⁻⁸ mol/L, 10⁻⁸ mol/L respectively). Once rhythmic contractions had been achieved cumulative additions of cystamine (Sigma-Aldrich; 10⁻⁸ to 10⁻² mol/L), mono-dansylcadaverine (MDC) (Sigma-Aldrich; 10⁻⁸ to 10⁻⁴ mol/L), cysteamine (Sigma-Aldrich; 10⁻⁹ to 10⁻² mol/L), putrescine (Sigma-Aldrich; 10⁻⁸ to 10⁻⁴ mol/L) were added at 25 minute intervals. Contractile activity was determine by activity integral measured from 20 minutes time periods for each drug addition and analysed along with time-matched control myometrial strips under the same conditions but with vehicle additions (physiological salt solution).

### Calcium Mobilisation Assay:

Myometrial smooth muscle cells were grown in DMEM (Dulbecco's Modified Eagle Medium; Sigma-Aldrich) supplemented with 10% fetal calf serum, L-glutamine and 0.2% pen/strep at 37° C. 5% CO₂, following dissociation in 2 mg/ml collagenase (Sigma-Aldrich). Once confluent cells were plated on black-walled flat-bottomed sterile 96-well plates (Costar) at a concentration of 10⁵ cells/ml and in the same media makeup and again grown to confluence. Once confluent, cells were incubated in 100 μl media/well containing 2.5 mM probenecid (Sigma-Aldrich), 2.3 μM Fluo-4 (invitrogen) and 0.023% pluronic acid (Invitrogen) for one hour in the dark at 37° C. 5% CO₂. Cells were then washed twice with phosphate buffered saline and finally incubated in 100 μl/well PSS (Chanrachakul, Broughton-Pipkin et al., Am J Obstet Gynecol 192(2): 458-63, 2005) containing 2.5 mM probenecid with or without 10⁻⁶ mol/L cystamine, MDC or cysteamine for 10 minutes. Fluorescent readout was then recorded on FlexStation (Molecular Probes) over a 200 second period with oxytocin addition at 15 seconds (10⁻¹¹ to 10⁻⁵ mol/L) and ionomycin, to measure maximal potential response, at 150 seconds (10⁻⁶ mol/L; Sigma-Aldrich).

### Results

### Example 1

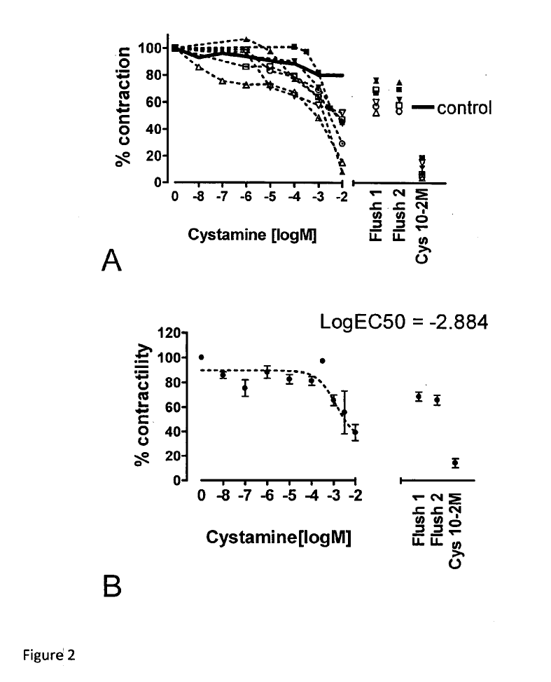

Oxytocin-induced contractions of human myometrial tissue are inhibited by the tissue transglutaminase II inhibitor cystamine in a dose-dependent manner (FIG. 2). At a concentration of 10⁻²M, cystamine reduces contractility to 14.2±3.7% of untreated control myometrium. Following removal of cystamine from the tissue by washing, oxytocin-induced contractions of the myometrium return to control levels.

### Example 2

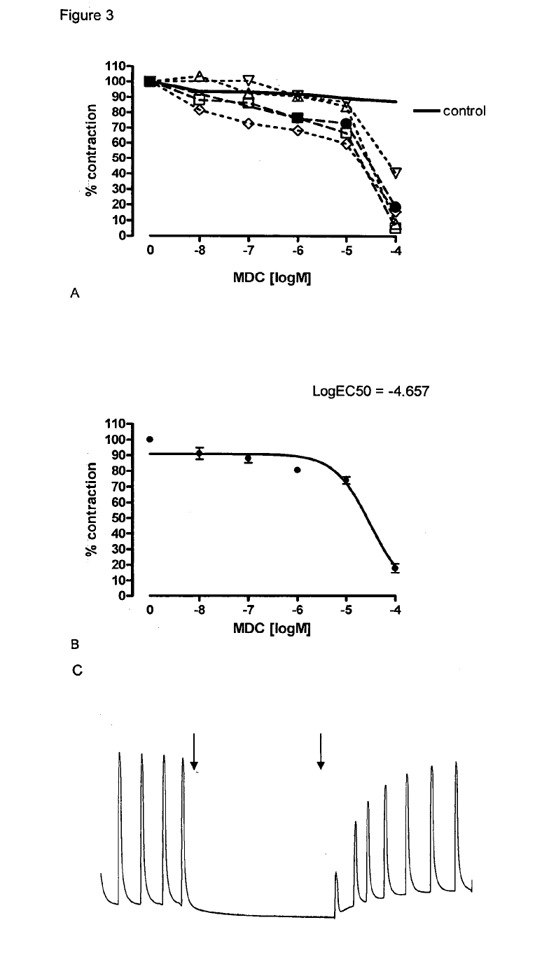

Oxytocin-induced contractions of human myometrial tissue are inhibited by the tissue transglutaminase II inhibitor Monodansylcadaverine (MDC) in a dose-dependent manner (FIG. 3). At a concentration of 10⁻⁴M, MDC reduces contractility to 17.8%±6.2% of untreated control myometrium. Following removal of MDC from the tissue by washing, oxytocin-induced contractions of the myometrium return to control levels.

### Example 3

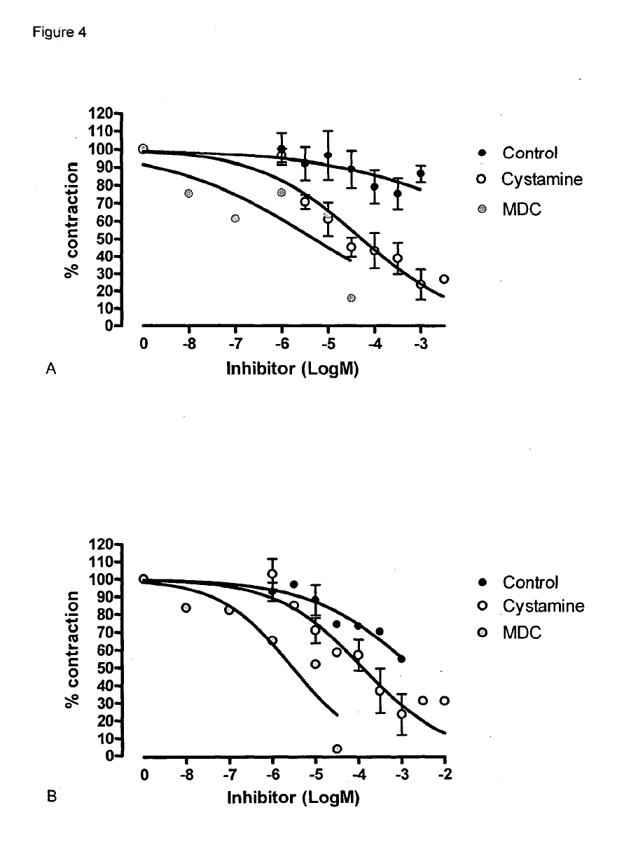

The tissue transglutaminase inhibitors cystamine and MDC also attenuate contractions induced by both bradykinin and phenylephrine (FIG. 4). This indicates that tissue transglutaminase inhibitors act to inhibit the contractile ability of the tissues generally rather than affecting a single agonist stimulated pathway.

### Example 4

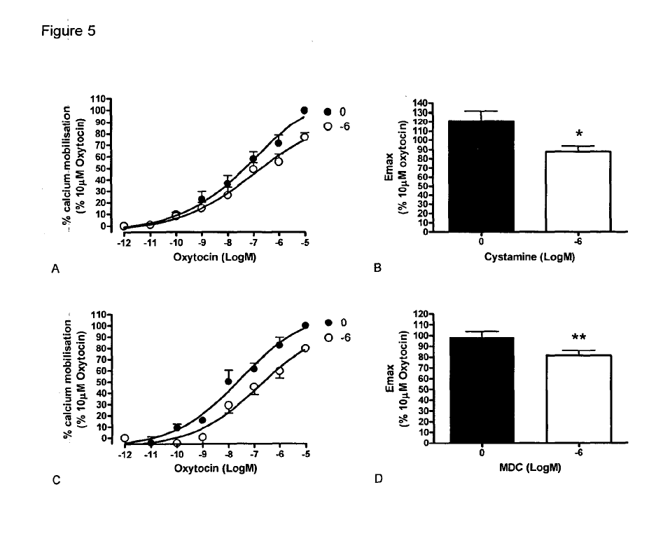

Oxytocin stimulated calcium mobilisation, as measured by a fluorescent calcium indicator in a cell-based assay, is affected by incubation with tissue transglutaminase inhibitors (FIG. 5). Maximal calcium mobilisation is significantly reduced after incubation with both cystamine and MDC at 10⁻⁶M.

### Example 5

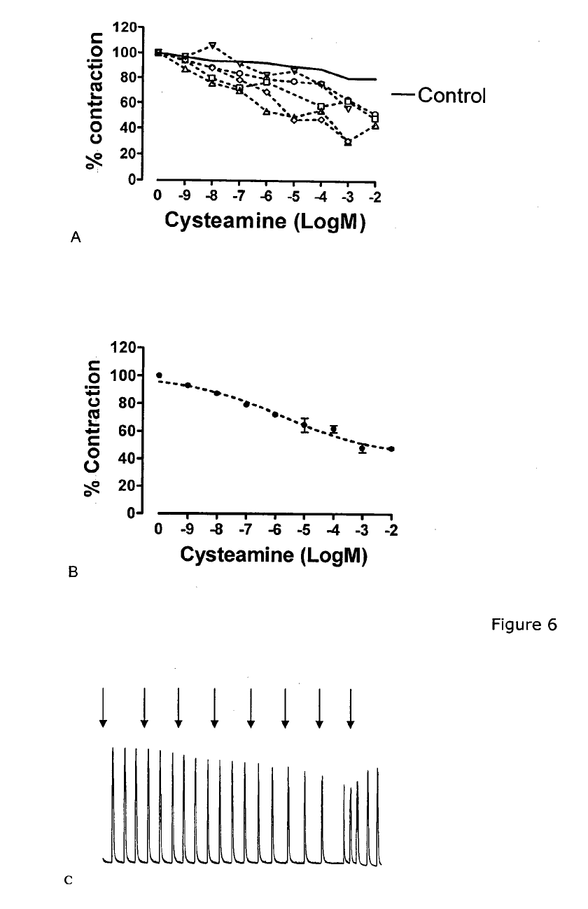

Oxytocin-induced contractions of human myometrial tissue are inhibited by cysteamine, the licensed metabolite of cystamine, in a dose-dependent manner (FIG. 6). Following removal of cysteamine from the tissue by washing, oxytocin-induced contractions of the myometrium began to return to control levels.

### Example 6

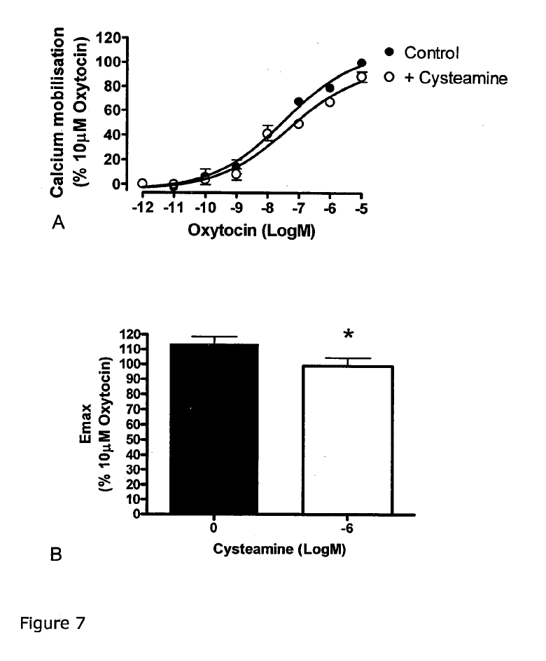

Oxytocin stimulated calcium mobilisation, as measured by a fluorescent calcium indicator in a cell-based assay, is affected by incubation with cysteamine, the licensed metabolite of cystamine (FIG. 7). Maximal calcium mobilisation is significantly reduced after incubation with cysteamine at 10⁻⁶M.

### Example 7

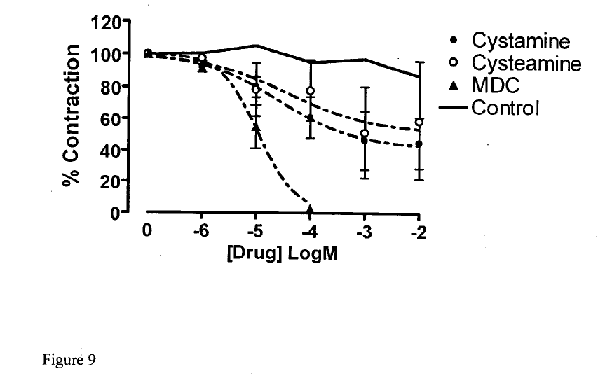

The data in FIG. 9 shows that spontaneous contractions in myometrial strips are inhibited by cystamine (n=4), cysteamine (n=3) and MDC (n=2) in a dose-dependent manner. This data demonstrates that the effect of TGII inhibition on contractions is independent of oxytocin receptor signalling.

1.-36. (canceled)

37. A method of treating or preventing a disorder originating in uterine contractions in a subject, said method comprising administering to the subject a therapeutically effective amount of an inhibitor of transglutaminase II (tTgII).

38. The method according to claim 37 wherein the inhibitor is an amine.

39. The method according to claim 38 wherein the amine is a member selected from a monoamine and a polyamine.

40. The method according to claim 39 wherein the polyamine is a diamine or triamine.

41. The method according to claim 39 wherein the amine is not a tetramine.

42. The method according to claim 39 wherein the amine is a member selected from monodansylcadaverine, biotincadaverine, putrescine, cadaverine, histamine, spermidine, cysteamine and cystamine.

43. The method according to claim 38 wherein the amine is a sulfur containing amine.

44. The method according to claim 43 wherein the sulfur containing amine comprises 1, 2 or 3 sulfur atoms.

45. The method according to claim 43 wherein the amine is an aminothiol.

46. The method according to claim 38 wherein the amine comprises one or more oxygen atoms.

47. The method according to claim 39 wherein the amine is not spermine.

48. The method according to claim 37 wherein the inhibitor is a competitive inhibitor of transglutaminase II (tTgII).

49. The method according to claim 37 wherein the disorder is preterm labor.

50. The method according to claim 37 wherein the amount of the inhibitor administered is sufficient to maintain a cessation of uterine contractions in the subject until such time as it is desirable to allow the contractions to resume in which case the amount of inhibitor administered is either reduced or stopped.

51. The method according to claim 37 wherein the inhibitor of transglutaminase II (tTgII) is administered together with another tocolytic agent in a separate, sequential or simultaneous manner.

52. A pharmaceutical formulation comprising an inhibitor of transglutaminase II (tTgII) in an amount effective to inhibit or counter the onset of uterine contractions, and a pharmaceutically acceptable carrier.

53. The pharmaceutical formulation according to claim 52, further comprising a tocolytic agent in addition to the inhibitor of transglutaminase II (tTgII).
